(19) United States
(12) Patent Application Publication
(10) Pub. No.: US 2026/0363887 A1
(43) Pub. Date: Nov. 19, 2026

(54) DEVICES AND METHODS FOR RELEASING AN IMPLANT ONTO A LUMENAL SURFACE

(71) Applicant: Restore Medical Ltd., Or Yehuda (IL)

(72) Inventors: Ori KOHN, Ramat Gan (IL); Tsur GENOSAR, Tel Aviv (IL)

(73) Assignee: Restore Medical Ltd., Or Yehuda (IL)

(21) Appl. No.: 19/477,483

(22) PCT Filed: Apr. 18, 2024
(86) PCT No.: PCT/IL2024/050393

§ 371 Date: Oct. 21, 2025

Related U.S. Application Data
(60) Provisional application No. 63/460,671, filed on Apr. 20, 2023.

(51) Int. Cl. A61F 2/966

(52) U.S. Cl. CPC A61F 2/966; A61F 2002/9665

(57) ABSTRACT

The instant invention, in some embodiments, describes delivery units for implants to a lumen surface. In some embodiments, a plurality of tabs are employed to engage an implant to each attachment point on an implant holder. The use of multiple tabs for each attachment point overcomes a possibility that a single tab may be spatially impeded from opening due to the shape of a lumenal space in which the implant is to be placed. At least one tab will necessarily be on a side opposite the lumenal wall and will disengage from an expanding implant, with a second tab necessarily falling off and allowing for complete separation between an implant and the delivery unit used in its deployment.

### RELATED APPLICATION/S

This application claims the benefit of priority of U.S. Provisional Patent Application No. 63/460,671 filed on Apr. 20, 2023, the contents of which are incorporated herein by reference in their entirety.

### FIELD AND BACKGROUND OF THE INVENTION

The present invention, in some embodiments thereof, relates to devices and methods for an implant in a lumen.

Delivery of stents and stent-based implants in arteries often requires delivery and release of implants in predetermine locations. In some locations, shape of an artery surface does not allow for facile implant release during surgical delivery. Curvature of a lumenal surface, for example, can prevent release of implant due to surface interference with implant release.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A delivery unit, comprising:
a capsule including an outer sheath and an inner implant holder having at least one attachment point for attaching an implant thereto;
an implant, comprising:
a first tab associated with a first circumferential location of said implant, wherein said first tab is sized and shaped for geometric engagement with said attachment point; and,
a second tab associated with a second circumferential location of said implant and sized and shaped for geometric engagement with said attachment point,
wherein said implant has a first, collapsed state within said sheath and a second, expanded state outside said sheath and wherein said first location and said second location are further circumferentially apart in said expanded state than in said collapsed state,
wherein said first tab and said second tab hold said implant to said attachment point in said first, collapsed, state, and move circumferentially apart in said second, expanded, state, thereby weakening an engagement of said tabs to said attachment point.

Example 2. The delivery unit according to example 1, wherein said implant includes at least one stent.

Example 3. The delivery unit according to example 1 or example 2, comprising one or more additional first tab and additional second tab, provided as pairs, wherein each tab is located on a unique position on said implant.

Example 4. The delivery unit according to any one of examples 1-3, wherein said implant holder comprises a retractable deployment arm and said at least one attachment point comprises a plurality of attachment points.

Example 5. The delivery unit according to example 2, wherein said at least one stent is made partially or wholly of Nitinol.

Example 6. The delivery unit according to example 2 or example 5, wherein said first tab and said second tab are associated with crown-shaped structures at an end of said stent.

Example 7. The delivery unit according to any one of examples 1-6, wherein said first tab and said second tab are identical in shape or mirrored.

Example 8. The delivery unit according to any one of examples 1-7, wherein said first tab and said second tab have a rounded feature.

Example 9. The delivery unit according to any one of examples 1-8, wherein said first tab and said second tab have a rectangular feature.

Example 10. The delivery unit according to any one of examples 1-9, wherein said first tab and said second tab face each other around said attachment point.

Example 11. The delivery unit according to any one of examples 1-10, wherein said first tab and said second tab are associated with different geometries of the attachment point.

Example 12. The delivery unit according to anyone of examples 1-11, wherein said capsule has an open end at a distal end thereof, for implant passage therethrough.

Example 13. The delivery unit according to any of example 12, wherein the distal end lacks a conical tip.

Example 14. The delivery unit according to any one of examples 1-13, comprising a guide that protrudes beyond the open distal end of said capsule, wherein said guide passes through said inner implant holder, and wherein said implant is crimped over said guide and within said capsule. Example 15. The delivery unit according to any one of examples 1-14, comprises a balloon, associated with said implant holder, and positioned adjacent to said first tab and said second tab, such that inflation of said balloon forces the separation of said first tab and said second tab.

Example 16. A method for releasing an implant from a delivery unit into a lumen, including: providing a crimped implant in a delivery unit comprised of an outer sheath and an inner implant holder, wherein said implant is attached to said implant holder with a first tab and a second tab associated with said implant and geometrically engaged to a same attachment point on said implant holder;
releasing said implant from said outer sheath at a position in a lumen;
allowing said implant to expand radially after said release; and,
allowing said first tab and said second tab to circumferentially move away from each other, so that at least one tab moves circumferentially away from said attachment point thereby weakening a coupling between said first tab and said second tab with said attachment point.

Example 17. The method according to example 16, wherein said lumen is an artery.

Example 18. The method according to example 16 or 17, further including fluoroscopic monitoring of positions of said first tab and said second tab after said allowing said implant to expand.

Example 19. The method according to any one of examples 16-18, wherein said implant is made, in part, of a self-expanding material.

Example 20. The method according to any one of examples 16-19, further including expanding a balloon associated with said implant holder after said releasing.

Example 21. The method according to any one of examples 16-20, wherein said releasing is initiated by moving said inner implant holder out of said outer sheath.22. The method according to any of examples 16-18, wherein said releasing is initiated by a rotation of said implant holder. Example 23. A delivery unit for a stent-like implant onto a lumenal surface, including:
a guide for directing insertion of an implant at a desired lumenal surface;
a capsule including an outer sheath and an inner implant holder having at least one attachment point for attaching an implant thereto, wherein said capsule has an open end for implant passage out of said capsule;
an implant, comprising:
a cylindrical self-expanding body sized to be implanted in a pulmonary artery.

Example 24. The delivery unit according to example 23, wherein said implant comprises:
a first tab associated with a first circumferential location of said implant, wherein said first tab is sized and shaped for geometric engagement with said attachment point; and,
a second tab associated with a second circumferential location of said implant and sized and shaped for geometric engagement with said attachment point,
wherein said implant has a first, collapsed state within said sheath and a second, expanded state outside said sheath and wherein said first location and said second location are further circumferentially apart in said expanded state than in said collapsed state,
wherein said first tab and said second tab hold said implant to said attachment point in said first, collapsed, state, and move circumferentially apart in said second, expanded, state, thereby weakening an engagement of said tabs to said attachment point.

Example 25. The delivery unit according to example 23 or 24, wherein said implant comprises:
a single tab associated with a circumferential location of said implant, wherein said single tab is sized and shaped for geometric engagement with a side of said attachment point; and,
wherein said implant has a first, collapsed state within said sheath and a second, expanded state outside said sheath,
wherein said single tab holds said implant to said inner implant holder in said first, collapsed, state, and moves away from said attachment point in said second, expanded, state, thereby weakening an engagement of said single tab to said inner implant holder.

Example 26. The delivery unit according to any one of examples 23 to 25, wherein the guide comprises a guide wire tube.

Example 27. The delivery unit according to example 26, wherein said guide wire tube extends beyond said open end.

Example 28. The delivery unit according to any one of examples 23 to 27, wherein the proximal end lacks a conical tip.

Example 29. The delivery unit according to any one of examples 23 to 28, wherein said open end has a cross-section area of at least 70% of a maximal outer diameter of said delivery unit. Example 30. A method for releasing an implant from a delivery unit into a lumen, including:
providing a crimped implant in a delivery unit comprised of a capsule and an inner implant holder, wherein said implant is attached to said implant holder with a single tab associated with said implant and geometrically engaged to a side of an attachment point on said implant holder;
releasing said implant from said capsule at a position in a lumen;
allowing said implant to expand radially after said release; and,
allowing said single tab to circumferentially move away from said attachment point, so that said single tab moves circumferentially away from said attachment point thereby weakening a coupling between said single tab with said attachment point.

Example 31. The method according to example 30, further including rotating a guide wire tube associated with said implant holder.

Example 32. The method according to example 30, comprising one or more additional single tabs, wherein each tab is associated with a unique position on said implant.

Example 33. A delivery unit, comprising:
a capsule having an inner volume defined by a wall of an outer sheath and an inner implant holder having at least one attachment point for attaching an implant thereto;
an implant, comprising:
a first tab associated with a first circumferential location of said implant, wherein said first tab is sized and shaped for geometric engagement with said attachment point; and,
a second tab associated with a second circumferential location of said implant and sized and shaped for geometric engagement with a same or a different attachment point,
wherein said implant has a first, collapsed state within said sheath and a second, expanded state outside said sheath and wherein said first location and said second location are further circumferentially apart in said expanded state than in said collapsed state,
wherein said first tab and said second tab hold said implant to said inner implant holder in said first, collapsed, state, and move circumferentially apart in said second, expanded, state, thereby weakening an engagement of said tabs to said inner implant holder.

Example 34. A delivery unit, comprising:
a capsule including an outer sheath and an inner implant holder having at least one attachment point for attaching an implant thereto;
an implant, comprising:
a single tab associated with a circumferential location of said implant, wherein said single tab is sized and shaped for geometric engagement with a side of said attachment point; and,
wherein said implant has a first, collapsed state within said sheath and a second, expanded state outside said sheath,
wherein said single tab holds said implant to said inner implant holder in said first, collapsed, state, and moves away from said attachment point in said second, expanded, state, thereby weakening an engagement of said single tab to said inner implant holder.

Example 35. The delivery unit according to example 34, comprises one or more additional single tabs, wherein each tab is associated with a unique position on said implant.

Biological and biomedical terms such as artery, lumen, stent, pulmonary, ventricular, interventricular, septum, and tissue may have their generally understood meanings in the medical arts.

Unless otherwise defined, all technical and/or scientific terms used herein may have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Some embodiments of the present invention may be described below with reference to flowchart illustrations and/or block diagrams of methods, apparatus (systems) and products according to embodiments of the invention. The instant invention, in some embodiments may be practiced with one or a plurality of elements or steps as described herein. It is understood that the instant invention may have application to other implants and or physiological insertion locations and is described for a stent-like insertion in a pulmonary artery solely for the purpose of clarity.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. Similar elements may have the same number, advanced by multiples of 100.

In the drawings:

FIG. 1A & FIG. 1B are schematic representations of an existing implant delivery system; FIG. 1C shows a version of a prior art implant delivery system;

FIG. 2 is a flowchart of an exemplary method, according to some embodiments of the invention;

FIG. 3 is a picture of an implant according to some embodiments of the invention;

FIG. 4A is a picture of a crimped implant in a delivery sheath and attached to an implant holder according to some embodiments of the invention;

FIG. 4B is a picture showing the relative position of tabs to implant holder during expansion of implant;

FIG. 5 is a picture of an implant delivery system according to some embodiments of the invention;

FIG. 6A & FIG. 6B are schematic representations of attachment tabs and an attachment point according to some embodiments of the invention;

FIG. 6C shows schematic views of possible shapes of mirror-image tab pairs;

FIG. 6D shows schematic views of possible shapes of attachment points;

FIG. 6E shows tabs around an attachment point for a device loaded into a catheter;

FIG. 7 is a schematic representation of an exemplary implant delivery unit, according to some embodiments of the invention;

FIG. 8A is a schematic representation of an exemplary implant delivery unit with implant deployment arm extended in a lumen;

FIG. 8B is a cross-sectional view of the schematic representation from FIG. 8A;

FIG. 9 is a schematic representation of an exemplary implant delivery unit with implant expanded outside of an implant capsule, according to some embodiments of the invention;

FIG. 10 is a schematic representation of an exemplary expanded implant implanted in a lumen, according to some embodiments of the invention;

FIG. 11A & FIG. 11B are schematic representations of an exemplary alternate tab pair, according to some embodiments of the invention;

FIG. 12A & FIG. 12B are schematic representations of forces related to separation of tabs by implant expansion, according to some embodiments of the invention;

FIG. 13A is a schematic representation of an exemplary balloon-based implant delivery system, according to some embodiments of the invention;

FIG. 13B is a schematic representation of an expanded balloon in a balloon-based implant delivery system, according to some embodiments of the invention;

FIG. 14 is a schematic representation of a delivery unit as per an embodiment of the invention;

FIG. 15 is a schematic representation of a guidance system used to delivery an implant capsule to a lumen according to an embodiment of the invention;

FIG. 16 shows a schematic representation of a delivery system without a nose cone ready for deployment;

FIG. 17 shows a flowchart for a method of the invention;

FIG. 18A & FIG. 18B show schematic views of single tab delivery systems with a nose cone unit; and,

FIGS. 19A-19D show schematic views of a self-expanding implant and release of tabs from associated delivery unit.

### DESCRIPTION OF SPECIFIC EMBODIMENTS OF THE INVENTION

The present invention, in some embodiments thereof, relates to delivering an implant in a lumen, and, more particularly, but not exclusively, to using a pair of tabs for each attachment point to allow for more facile delivery of implant, especially in curved implantation positions.

### Overview

An aspect of some embodiments of the invention relates to devices and methods for delivering a medical implant by using securing tab pairs that can move away from each other and/or an associated attachment point during an expansion of the implant in the body. The pairs may generally be located on different crowns associated with an implant and may either be held together or not. In some embodiments, the device allows for the precise delivery of an arterial implant with facile release of the expandable implant from its delivery system. In some embodiments, pairs of tabs are engaged to an implant holder at an attachment point to hold a crimped version of the implant in place prior to deployment. In some embodiments, the pairs of tabs release the implant holder concomitant or shortly after expansion of implant in situ in a lumen such as an artery. In some embodiments, a balloon is employed to release tab pairs. In some embodiments, additionally or alternatively, a physician may employ fluoroscopic monitoring to determine tab pair separation.

An aspect of some embodiments of the invention relates to facile separation of a medical implant from its delivery unit in the body. In some embodiments, an implant or a plurality of implants may be implanted in pulmonary arteries (PAS) by a designated delivery system during a transfemoral or transjugular venous procedure under fluoroscopy guidance. The instant invention, in some embodiments, overcomes a problem frequently encountered in stent and other implant procedures. An implant is routinely released from an implant delivery system. After implant release, there must be complete separation of implant and typically an deployment arm (also referred to herein as implant holder and/or engagement arm) that pushes implant out of a delivery vehicle and/or otherwise engages the implant during delivery. In some cases, for example, when the implant is released in a curved portion of a lumen and/or is delivered using a more rigid guidewire, the release tab connecting the deployment arm and the implant can at times fail to release due to physical blocking by the lumen surface (e.g., being trapped between the implant and the vessel wall. The result can be unintended movement or misplacement of the implant due to failed separation of the now-expanded implant and the deployment arm.

Upon crimping and loading of an implant according to some embodiments of the instant invention within a delivery unit, “half-tabs” or first and second tabs come together to form a full tab and engage the implant to an deployment arm (or implant holder). Upon deployment from an outer sheath structure and/or other release, the two tabs will have a separation force from the expanding implant to distance one-half tab from its other half and thereby weaken their interaction with associated attachment point, thus effecting full implant release. In some cases, even if a tab is not released, when the delivery system is moved (e.g., retracted), the engagement of the single tab will not be enough to move the implant (which is expanded against the vessel walls) in a manner which might damage the walls. An implant that does not fully separate may be dragged through a vessel or might be misplaced in its location, possibly requiring further surgery for correction.

An aspect of some embodiments of the invention relates to an open-ended delivery system. In some embodiments of the invention, a delivery system for a vascular implant includes a capsule which is optionally open at a distal end thereof, optionally, other than a guide, such as a guide wire tube extending past an end of the capsule. Herein, a guide wire tube refers to an elongated element, that comprises a wall, which defines a lumen along the longitudinal axis thereof. In some embodiments, the capsule comprises an inner volume that is defined by a wall of an outer sheath. Such a delivery system may be usable in any size blood vessel or other biological vessel. A potential advantage of such a design is when delivering a stent with a narrowing section. In a delivery system with a closed leading edge, the leading edge represented as a nose cone or tip may need to be retracted back past the implant after deployment of the implant. In such retraction, the nose cone may either be trapped between the implant and lumen wall or get entangled with the narrowing of the implant, both generally bad outcomes, which may be avoided by not having any leading component beyond a guide wire tube and a retractable, outer capsule. Trapping and entanglement may cause dislodgment of the implant when such an entangled cone or tip are retracted.

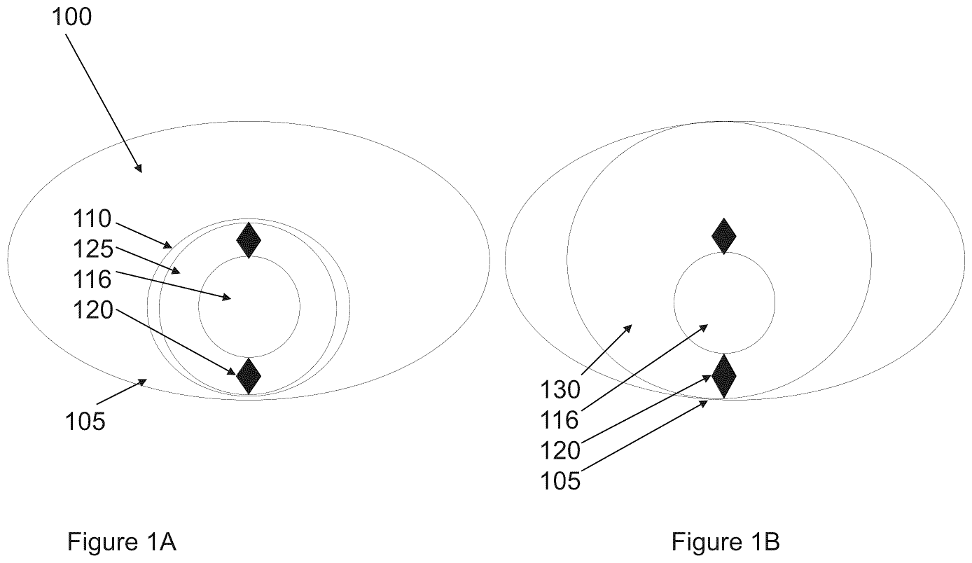

Referring now to the drawings, attention is turned to FIG. 1A which relates to a device without tab pairs. FIG. 1A shows a schematic view of a cross-sectional view of a prior art implant delivery system 100. A prior art implant delivery system 100 is located in a curved portion of a lumen 105. The prior art implant delivery system 100 includes a delivery sheath which defines a capsule 110, a retractable deployment arm 116, a connector tab 120, and an implant 125 in a retracted state held to deployment arm 116 via the connector tab 120. (Deployment arm 116 can also be referred to herein as an implant holder, engagement arm, and/or delivery arm). In some embodiments, deployment arm 116, is mounted on guide 115, such as a guide wire tube, optionally, the guide is threaded through deployment arm 116. In FIG. 1A, the prior art implant delivery system 100 is in place for delivery of crimped implant 125 in lumen 105. Attention is turned to FIG. 1B. Attempted release of an expanded implant 130 fails due to singular connector tab 120 being hindered by its close physical proximity of a curved portion of lumen 105. As connector tab 120 does not have the space to move to release expanded implant 130 from deployment arm 116, the expanded implant 130 remains connected to the deployment arm 116. If a physician believes that he/she has delivered the expanded implant 130 to the proper location in the lumen 105, then he/she may simply drag the still-connected expanded implant 130 during removal of the delivery sheath and deployment arm 116. The expanded implant 130, which can be larger than the lumen 105 itself, may thus be mispositioned and in need of additional procedure to move it back into place. Spatial hindrance in lumen 105 of full connector tab 120 release can lead to significant medical issues that might require additional surgery to correct.

The instant invention, in some embodiments, addresses the weakness of traditional single connector tabs 120. In some embodiments of the instant invention, a pair of “half-tabs” or “tab pairs”, rather than a single “full” connector tab 120 are employed. A potential advantage of connector tab pairs, as herewith described, is that one of the connector tabs, in a deployed device, is typically on a side away from the curved lumen 105 or the like. Once one tab separates from a side of an attachment point 118 associated with deployment arm 116 (e.g., implant holder 116), then perforce the second tab will disassociate, and the implant may be delivered fully free of its delivery apparatus. In other embodiments, a single tab is employed, wherein said single tab only sits on one side of a connection point (multiple such single tabs may be provided). FIG. 1C shows a prior art delivery system 100 with a single connector tab 120 that is attached to an deployment arm 116 on at least two sides of a connection point 118 of the deployment arm 116. Connector tab 120 may not fully separate from deployment arm 116 (e.g., from connection point 118 thereof) as previously described.

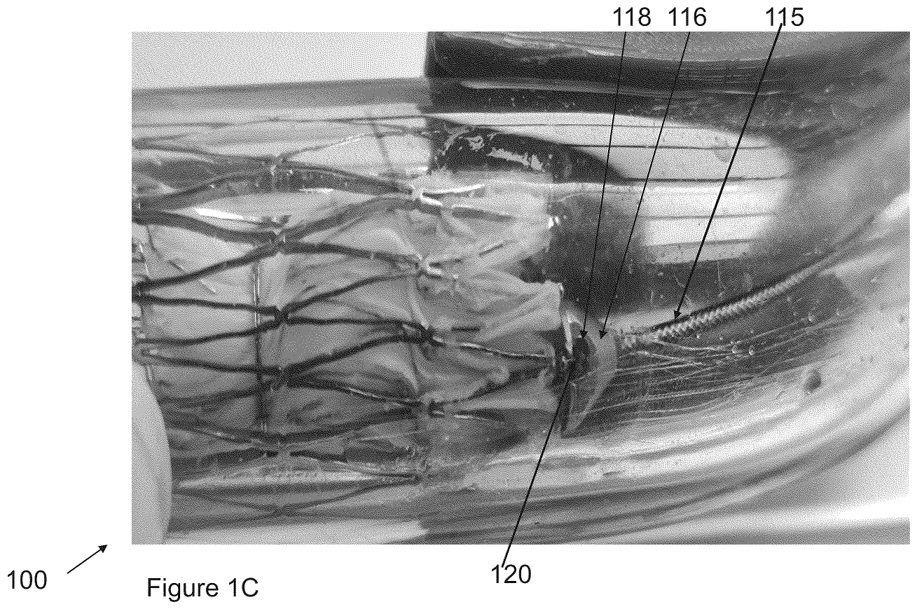

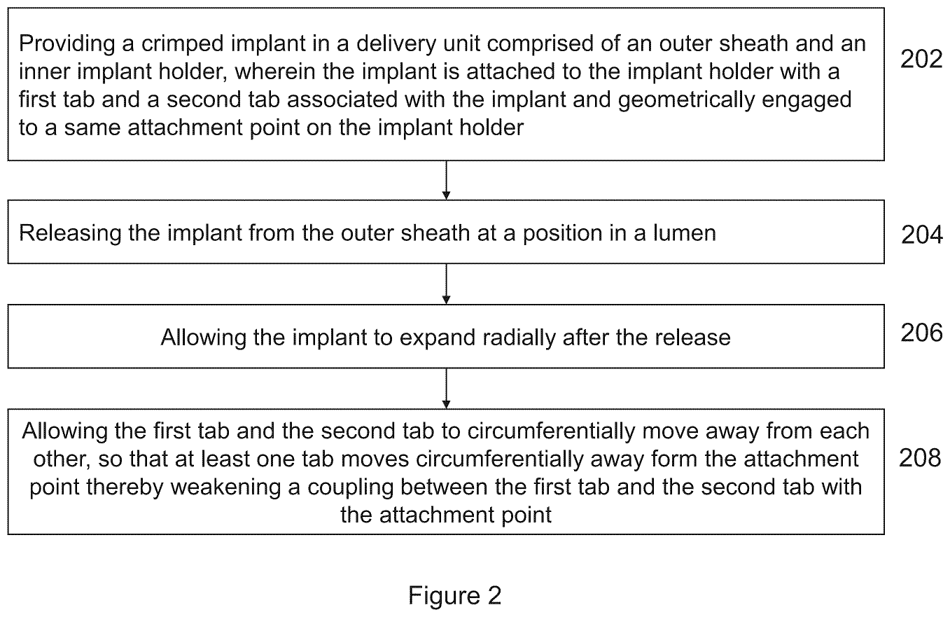

Attention is turned to FIG. 2. FIG. 2 illustrates a flowchart of an exemplary method, according to some embodiments of the invention. In some embodiments, an exemplary method for delivering an implant to a lumen, comprising: providing a crimped implant in a delivery unit comprised of an outer sheath and an inner implant holder, wherein the implant is attached to the implant holder with a first tab and a second tab associated with the implant and geometrically engaged to a same attachment point on the implant holder (202); releasing the implant from the outer sheath at a position in a lumen (204); allowing the implant to expand radially after the release (206); and, allowing the first tab and the second tab to circumferentially move away from each other, so that at least one tab moves circumferentially away from the attachment point thereby weakening a coupling between the first tab and the second tab with the attachment point (208).

In some embodiments, the method is applied in a lumen. In some embodiments, the lumen is a lumen of an artery. In some embodiments, the method includes fluoroscopic monitoring of positions of the first tab and the second tab after the allowing the implant to expand. In some embodiments of the invention, one or more radiopaque markers are provided on the implant crown and/or on one or more tabs, so that spacing between them can be used to identify device deployment. Optionally or additionally, a radiopaque marker is provided on the deployment arm. Optionally or additionally, deployment is tracked by identifying (e.g., on an x-ray image) if the deployment arm is against one side of the expanded stent/implant (e.g., and the stent may be pushed against the wall). Optionally or additionally, an x-ray image can be taken (e.g., by rotating a c-arm x-ray imager to correct orientation) to see if the two tabs at the location where the deployment arm meets the stent are separated or not. If the stent is fully opened (the crown is fully open and separated, then at most one tab is trapped between the deployment arm and the vessel lumen wall. As per the design in accordance with some embodiments of the invention, such engagement of the deployment arm by such tab is not robust enough to cause movement of the stent when the deployment arm is retracted.

In some embodiments, the implant is made, in part, of a self-expanding material. In some embodiments, the self-expanding material includes Nitinol. In some embodiments, there may be an additional (or alternative) expanding of a balloon associated with the implant holder after the releasing. In some embodiments, the releasing is initiated by moving the inner implant holder out of the outer sheath.

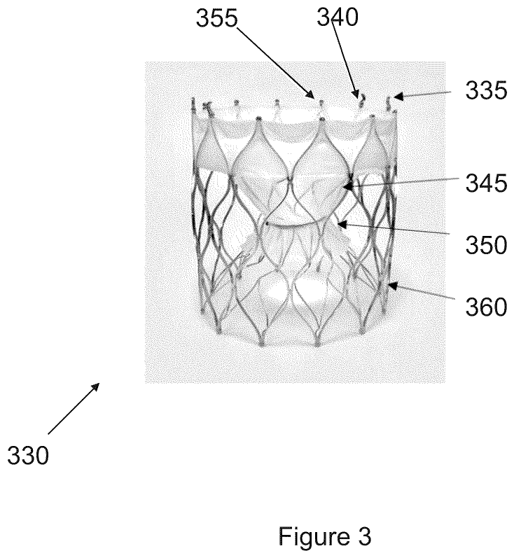

Referring now to FIG. 3, showing a schematic representation of a non-limiting example of an expanded implant 330 used in arterial procedures prior to crimping (not shown). The expanded implant 330 includes a first tab 335 and a second tab 340 located at an end of the implant 330 and are adapted to attach to an implant holder (not shown) at an attachment point during implant crimping. The specific implant 330 shown in FIG. 3 is optionally used to treat heart failure by modifying pressures in a predetermined location in the heart. The implant 330 includes a covered inner frame 345 to create a constricted channel with a constriction mechanism 350 to hold the constricted configuration. The implant 330 is made of at least one inner stent inside an outer stent 360. The first tab 335 and second tab 340 sit above adjacent crown-shaped structures 355 at one end of the outer stent 360. Another pair of tabs is located on the side opposite. The crimping process brings the first tab 335 and second tab 340 sitting on nearby crowns 355 closer together so as to form a full tab around a joining point (not shown). There are generally twelve crown-shaped structures 355 on an end of the implant 330. This number is not absolute and the number of crowns can be larger or smaller. The percentage of crowns with tab features may be between 10 and 80%, for example. Generally, tabs may be found on 25% of crowns. Tabs may also alternatively or additionally be found on valves.

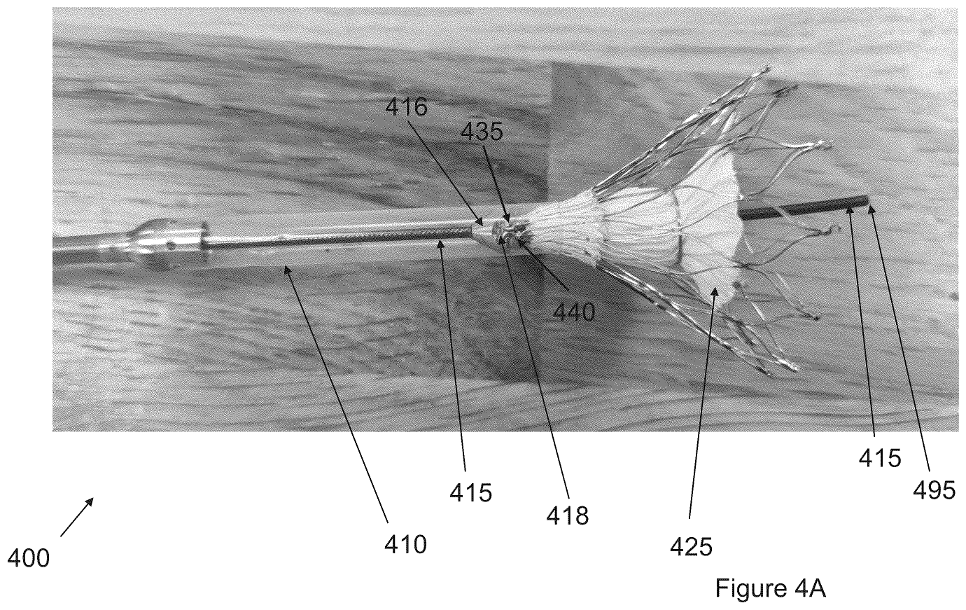

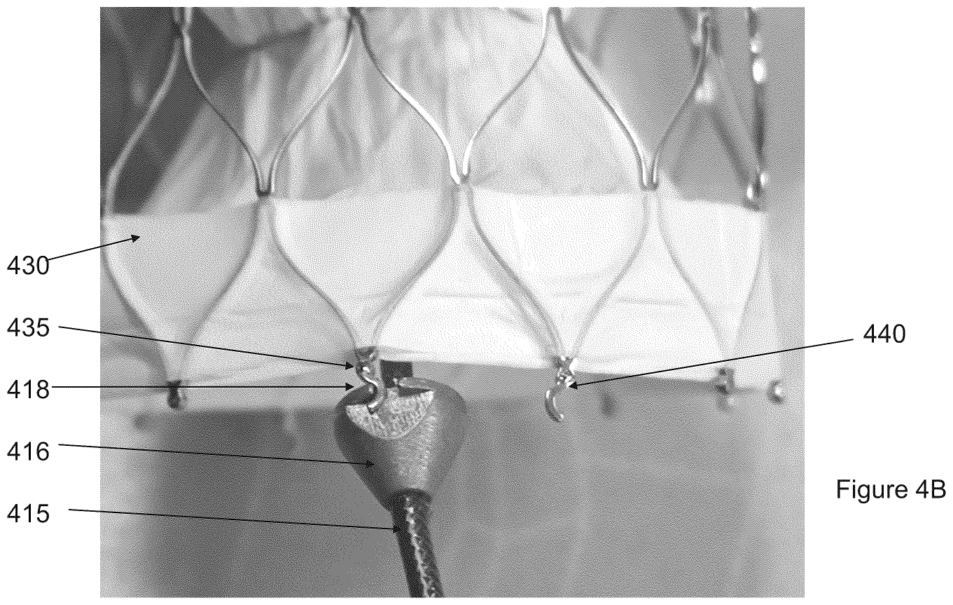

Referring now to FIG. 4A, showing a view of a crimped implant 425 partially deployed outside of its catheter capsule 410. In some embodiments, capsule 410 comprises an inner volume that is defined by a wall of an outer sheath Capsule 410 (e.g., outer sheath) may generally be made of any material, though plastics are preferred, and PET is particularly preferred due its flexibility and ability to be sterilized. The implant 425 may be released up to 50% and still be brought back into the capsule 410 if need be. Tabs 435 and 440 are visible and are attached to an attachment point 418 associated with a deployment arm 416. One may note that at the distal end 495 of the delivery system 400 there is no nose cone or tip present. Deployment arm 416 is connected and/or mounted on guide 415, which optionally comprises an inner lumen along the longitudinal axis thereof. Herein the guide can also be referred to as a guide wire tube (as shown for example in FIG. 4A). It is noted that the guide cross-section is not limited to a circular shape and may have any other cross-section shape. Deployment arm 416 comprises a passageway for guide 415 to pass therethrough, such that deployment arm 416 is positioned over guide 415, as shown for example in the figure. In some embodiments, distal end 495 of delivery system 400 is defined by the distal end of guide 415, alternatively or additionally, distal end 495 of delivery system 400 is defined by the distal end of capsule 410.

Referring now to FIG. 4B, tab 435 is still temporarily associated with attachment point 418 on the deployment arm 416 while tab 440 is completely removed from its original position. Implant 430 expansion leads to tab 435 & 440 disassociation with deployment arm, which in turn allows for potentially safe and successful deployment of implant 430 fixed into a lumenal position. In some embodiments, after the implant expansion, guide wire tube 415 is retracted to remove the delivery system. This dual-tab delivery system has the potential advantage of reducing and/or avoiding the risk of implant dislodgment from the deployment site, during the system retraction. This applies whether both tabs 435, and 440 are disconnected from attachment point 418 or whether one of the tabs is still associated therewith. The connection between one tab (e.g., one tab of the tab pair) is potentially insufficient to pull (and dislodge) the implant during retraction of the delivery system. In some embodiments, the tabs (e.g., tabs 435, 440) are sized and/or shaped such that the withdrawal of guide 415 (e.g. the withdrawal of delivery system 400) while one of the tabs is still associated with attachment point 418 results in the disconnection of said tab.

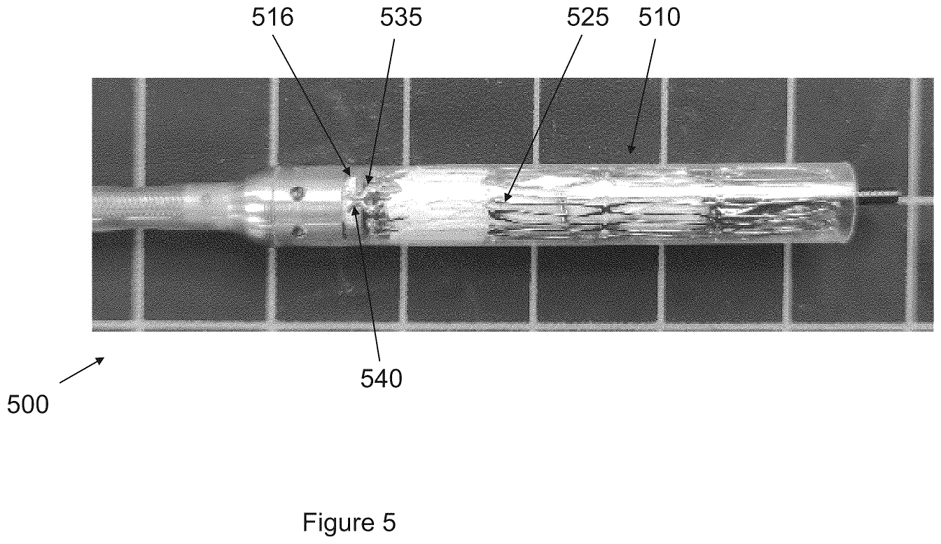

Referring now to FIG. 5, showing a view of an implant delivery unit 500 with compacted implant 525 inside a nylon outer sheath (or capsule) 510. The implant 525 is ready for delivery to patient where Nitinol of the implant 525 will expand after release of implant from the outer sheath (or capsule) 510 and exposure of the implant 525 within the lumen. First tab 535 and second tab 540 are visible through the outer sheath 510 and are in a closed arrangement to hold the implant 525 to the implant holder 516 until full deployment of the implant 525 outside the capsule 510.

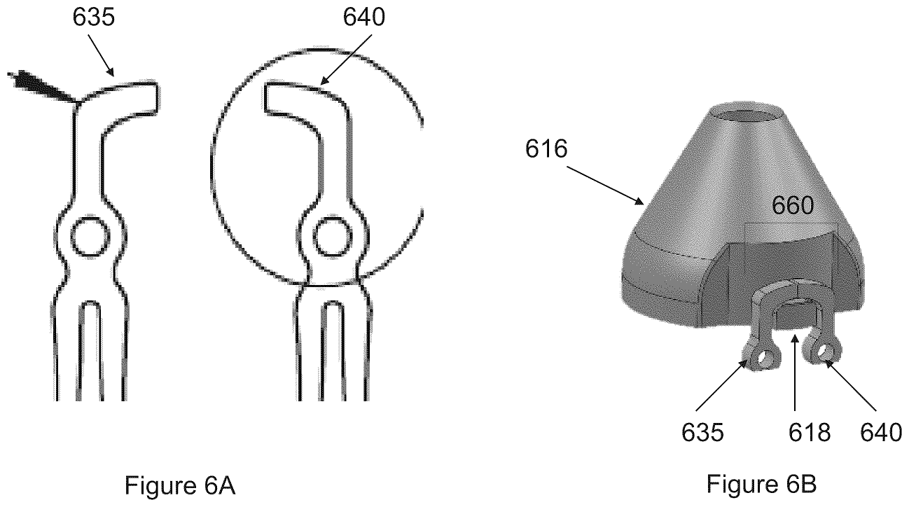

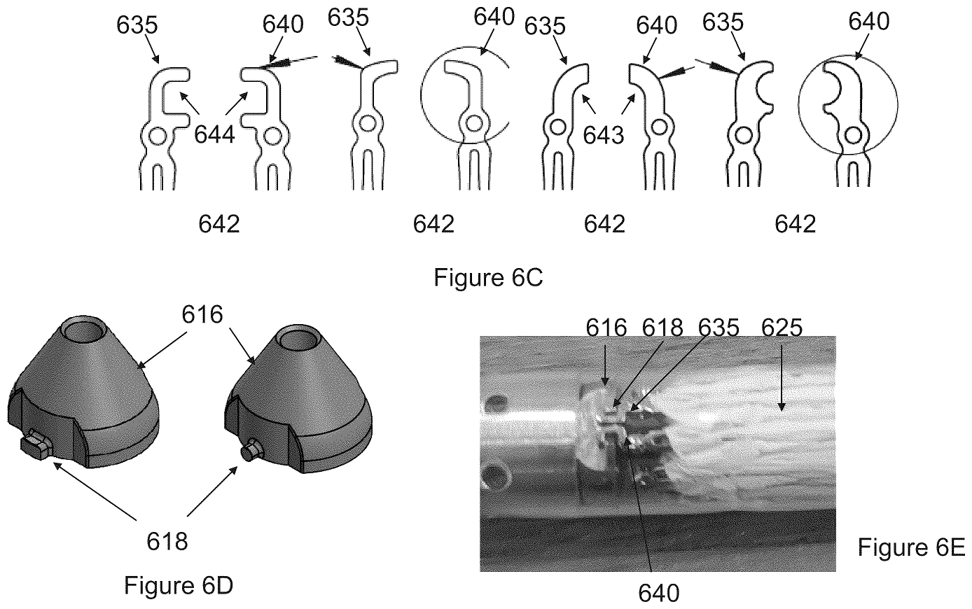

Referring now to FIG. 6A, showing a schematic representation of tabs according to an embodiment of the invention. A first tab 635 and a second tab 640 are optionally mirror images of one another. Each tab is adapted to engage an attachment point associated with an implant holder (not shown) only on a single side of the attachment point (such that each tab is adapted to engage a different side of the attachment point). Thus neither first tab 635 nor second tab 640 is adapted to reliably engage an attachment point alone, but together they can engage one or more geometries of the attachment points which potentially leads to a secure association of a crimped implant (not shown) with an implant delivery unit. FIG. 6B shows first tab 635 and second tab 640 both situated on either side of, and attached to, attachment point 618 associated with an implant holder 616. As can be seen in FIG. 6B, in some embodiments, neither tab is adapted to hold attachment point 618 alone, unlike the single tab shown in FIG. 1 (FIGS. 1C, 120). In FIG. 6B, first tab 635 and second tab 640 combine to form a whole-tab (660) which holds an implant (not shown) firmly to the implant holder 616. A particular feature of some embodiments of the invention is that when implant separates from attachment point 618, only either the first tab 635 or second tab 640 maximally can be obstructed by a lumen wall. As such, the other tab will be free to separate from the attachment point 618, and perforce, the obstructed tab will separate and the expanded implant will be fully free of the implant holder 616. Implant expansion forces first tab 635 and second tab 640 apart for full implant release, thus removing the risk of dragging the implant to an incorrect location.

FIG. 6C shows four pairs 642 of mirror image first and second tab pairs (e.g., each pair is defined by first and second tab 635, 640). The shapes are not limiting but rather offer additional views of potential tab shapes for application in the instant invention. In some embodiments, at least one of first and second tabs 635, 640 comprises a rounded feature, for example, feature 643, positioned at the contact with attachment point 618. The rounded feature potentially allows first and/or second tabs 635, 640 to slide along attachment point 618, upon expansion of the implant, while moving away from each other. In some embodiments, the rounded feature matches a rounded shape of at least a portion of attachment point 618.

In some embodiments, at least one of first and second tabs 635, 640 comprises a rectangular feature, for example, feature 644, positioned at the contact with attachment point 618. The rectangular feature potentially allows first and/or second tabs 635,640 to grab attachment point 618, having the potential advantage of reducing the risk of undesired release of first and/or second tabs 635,640 from attachment point 618, for example before reaching a desired deployment site and/or before implant expansion. In some embodiments, the rectangular feature matches a rectangular shape of at least a portion of attachment point 618.

FIG. 6D shows non-limiting attachment points 618 on an implant holder 616. FIG. 6E shows a crimped implant 625 attached via first tab 635 and second tab 640 to an attachment point 618 of an implant holder.

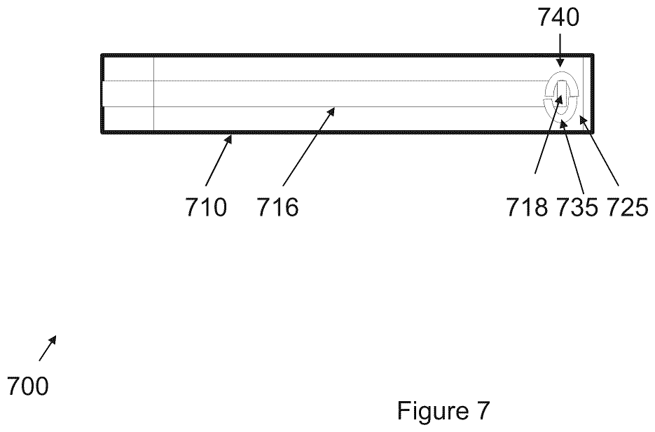

Referring now to FIG. 7, showing a schematic representation of an implant delivery unit 700 including an outer plastic sheath 710 (e. g capsule), a flexible inner implant holder 716, a crimped implant 725 having a first tab 735 and a second tab 740 secured during crimping around an attachment point 718 associated with the implant holder 716. FIG. 7 shows the implant delivery unit prior to a transfemoral venous procedure and prior to implant 725 delivery to a position on a lumen.

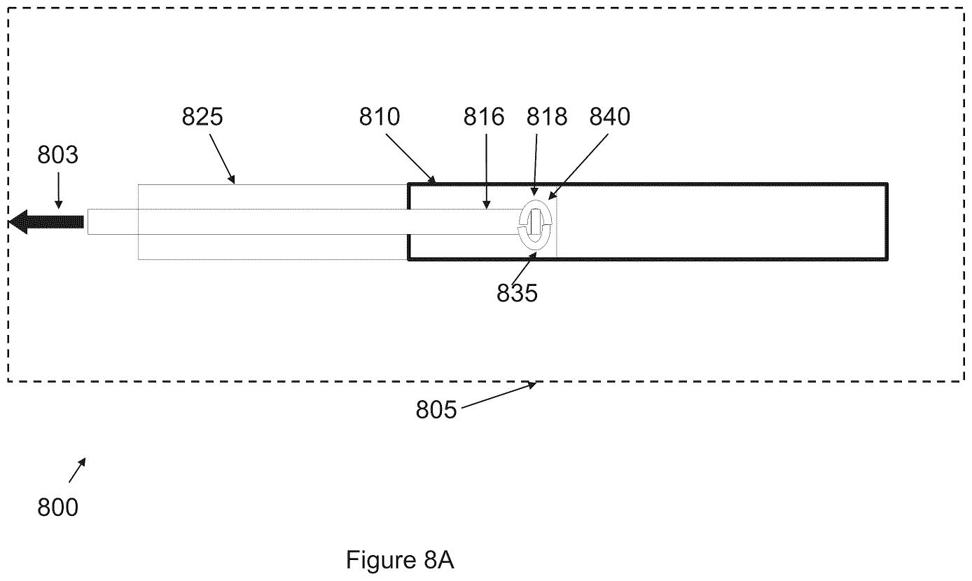

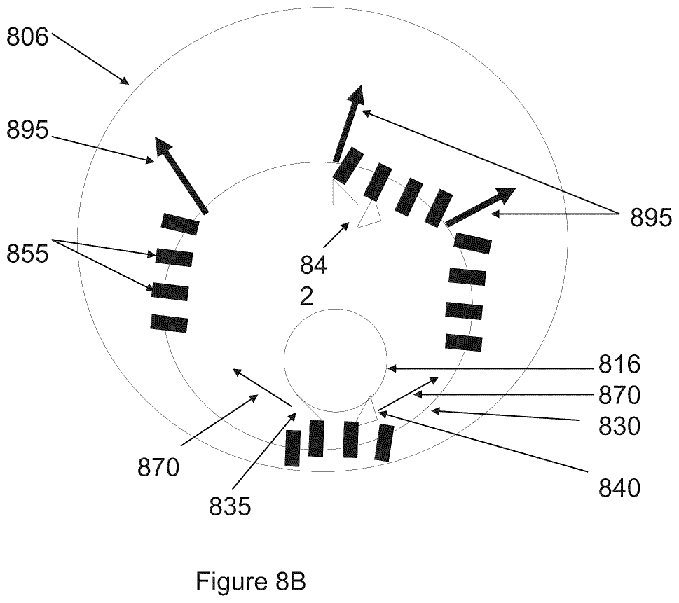

Referring now to FIG. 8A, showing a schematic representation of a view through a lumen 805 of an implant delivery unit 800 in which an implant holder 816 is partially ejected from an outer sheath/capsule 810 in proximity to a lumen 805 onto which an implant 825 will be placed. First tab 835 and second tab 840 are still fully attached to attachment point 818 at end of implant holder 816.

Referring to FIG. 8B which shows a schematic representation of a cross-sectional view of an expanding 895 implant 830 in a lumenal vessel 806. The lumenal vessel 806 may be, in a non-limiting example, an artery and the implant 830 may be a stent or stent-like device. In FIG. 8B, the implant 830 is expanding 895 radially away from an implant holder 816. Expanding 895 behavior is related to properties of Nitinol and other self-expanding materials. First tab 835 and second tab 840 are attached to the crown-shaped structure 855 at one end of the implant 830 while a second set 842 of tabs are already disconnected from the implant 830. First tab 835 and second tab 840 move away radially from implant holder 816 due to forces 870 generated by the expanding 895 implant 830 outside of its capsule (not shown). These forces 870 are large enough to cause complete separation of first tab 835 and/or second tab 840 from the attachment point on the implant holder 816 and allow for clean separation of implant 830 from the implant holder 816 and associated capsule. The free implant 830 will implant into the lumenal vessel 806 and remain in place.

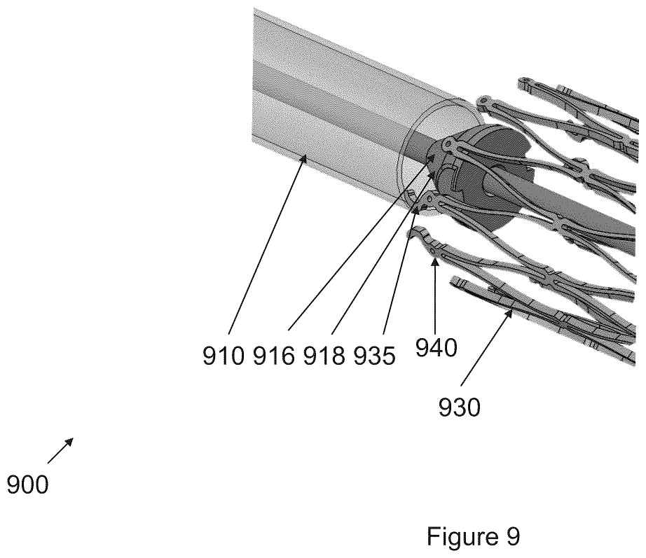

Referring now to FIG. 9, showing a schematic representation of a self-expanding implant 930 outside of the delivery unit 900 outer sheath 910 (e.g., capsule 910). The Nitinol in the implant 930 causes immediate expansion of implant 930 when the latter is freed from the restraint of a catheter capsule 910. Tab 935 and tab 940 have already been released from the attachment point 918 on the implant holder 916 and thus the implant is safely free of its delivery unit 900. Expansion of implant 930 can lead to a size greater than the vesicle in which it is deployed, thus guaranteeing its lack of movement away from the deployment site.

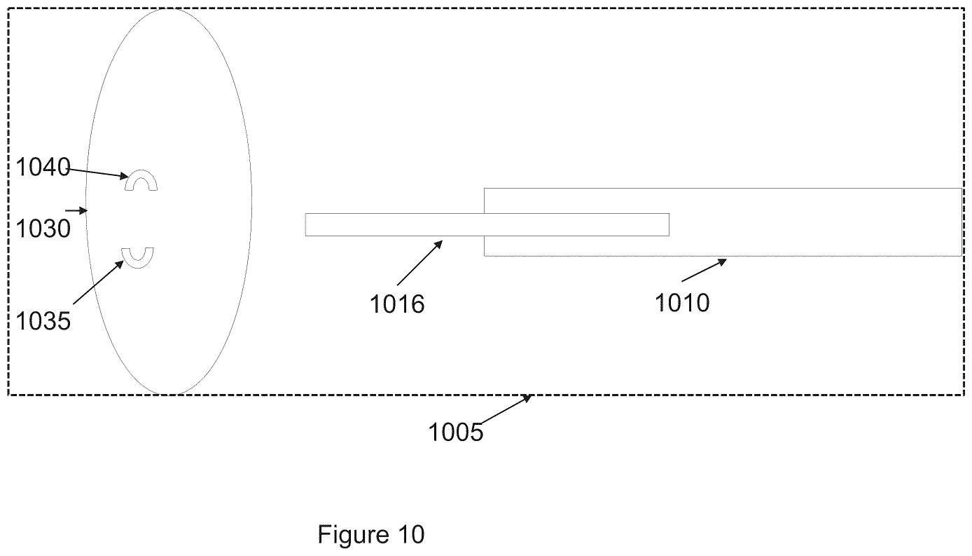

Referring now to FIG. 10, showing a schematic representation of a fully-expanded implant 1030 implanted into a lumen 1005. The view is again looking through the lumen 1005. First tab 1035 and second tab 1040 remain associated with implanted implant 1030 and outer sheath 1010 and inner implant holder 1016 may be safely removed without dragging or disturbing implant 1030. First tab 1035 and second tab 1040 are of no medical relevance when associated with the implant 1030 in the lumen 1005. The implant 1030 tends to fill the entire space of the lumen 1005 where it is located.

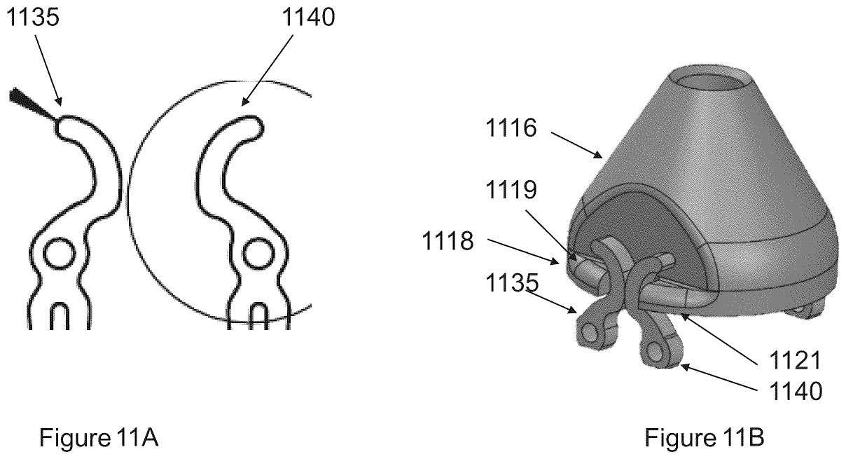

Referring now to FIG. 11A, showing a schematic representation of a second embodiment of a first tab 1135 and a second tab 1140 again having mirror symmetry as shown. In some embodiments, the attachment point is defined by a geometry, such as a protrusion, for example, attachment point 618 shown in FIG. 6B. In other embodiments, the attachment point, for example, attachment point 1118, comprises more than one geometry, such as more than one protrusion. FIG. 11B shows the instant embodiment of tabs and their association with attachment point 1118 (shown in FIG. 11B), which comprises a pair of distinct geometries (e.g., protrusions) 1119 and 1121. Each tab is associated with a different geometry, for example, first tab 1135 with geometry 1119 and second tab 1140 with geometry 1121 on the inner implant holder 1116. The pair of tabs are physically next to one another as shown schematically in FIG. 11B. Expansion of an associated implant (not shown for ease of viewing) leads to radial separation of first tab 1135 and second tab 1140 and their disassociation from the implant holder 1116. Forces generated by expanding Nitinol or similar implant material perforce separate adjacent tabs 1135 and 1140 and potentially lead to full separation of the tabs and associated implant with the implant holder 1116. It is understood that in some embodiments, a plurality of tab pairs are employed to secure an implant with an implant holder 1116 via a plurality of attachment points. In some embodiments, an expanding balloon in conjunction with a stiff implant can drive separation of tabs 1135 and 1140.

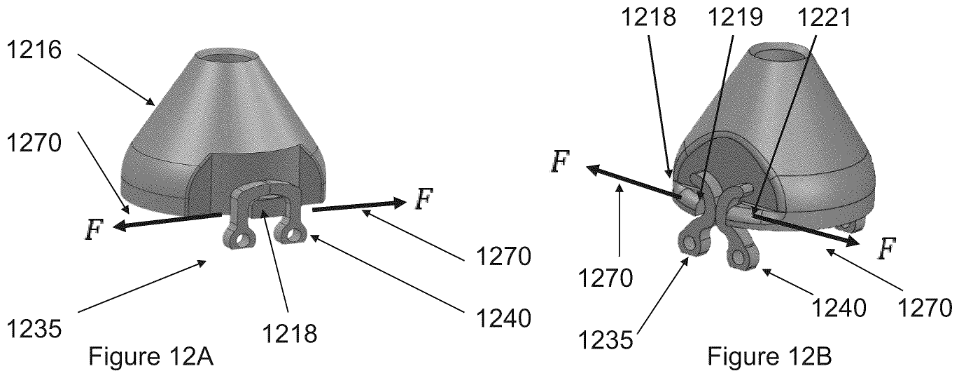

Referring now to FIG. 12A, showing a schematic representation of forces applied to first tab 1235 and second tab 1240 when both tabs are associated with attachment point 1218 defined by a single geometry (e.g., a single protrusion). Expanding implant (not shown) applies forces 1270 of an expanding circumference of the implant on the tabs to lead to their physical separation and distancing one from another. The self-expansion of the implant leads to the forced separation of the first tab 1235 and second tab 1240 from the attachment point 1218 on the implant holder 1216. The movement from a collapsed to an expanded state by virtue of the forces 1270 applied to first tab 1235 and second tab 1240 leads to greater circumferential separation of the tabs in the expanded state of the implant. FIG. 12B shows similar forces 1270 as applied to an alternative embodiment of the first tab 1235 and second tab 1240. In some applications, the guide of the delivery system (for example, guide 415, shown in FIG. 4B)may be rotated so as to further facilitate separation of first tab 1235 and/or second tab 1240 from attachment point 1218, which is defined by two geometries (e.g., two protrusions) 1218, 1221. One may note that since the first tab 1235 and second tab 1240 are not associated with the same geometry of attachment point 1218, the rotation allows the release of the tabs to guarantee full device deployment.

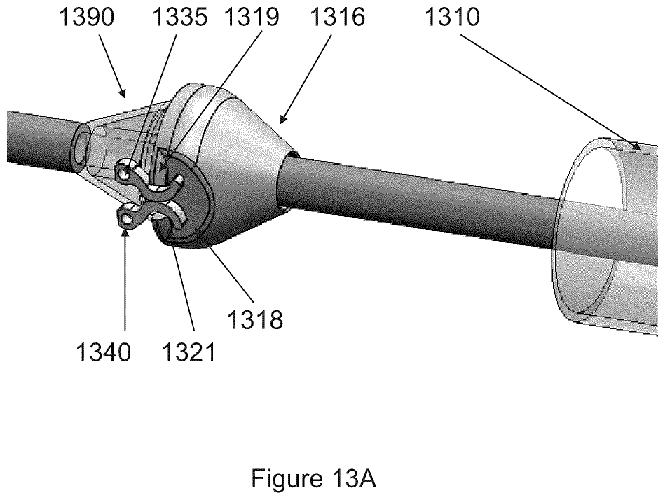

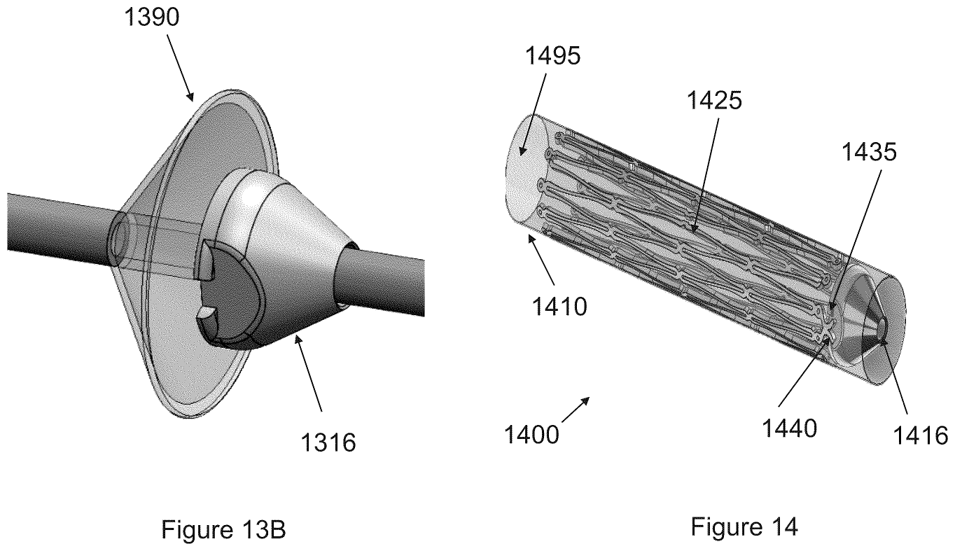

Referring now to FIG. 13A, showing a schematic representation of an embodiment of the invention employing a balloon 1390. The balloon 1390 is optionally located on a catheter (or guide-wire lumen) associated with the implant holder 1316. The implant holder 1316 is visible as extended from the external sheath 1310 prior to balloon 1390 inflation. In this non-limiting embodiment, first tab 1335 and second tab 1340 are associated with different geometries (e.g., protrusions) 1319 & 1321, of attachment pointassociated with the implant holder 1316. After the implant is fully deployed from the external sheath, inflation of a balloon 1390 may be employed to ensure that the first tab 1335 and second tab 1340 are forced to separate, thereby releasing the implant from the implant holder.

Referring now to FIG. 13B, showing a schematic representation of the embodiment associated with FIG. 13A in which the balloon (FIGS. 13B, 1390) has been inflated. Inflation of the balloon distances the first tab and second tab (not shown) from the implant holder 1316, which in turn ensures disengagement of the implant from the delivery system.

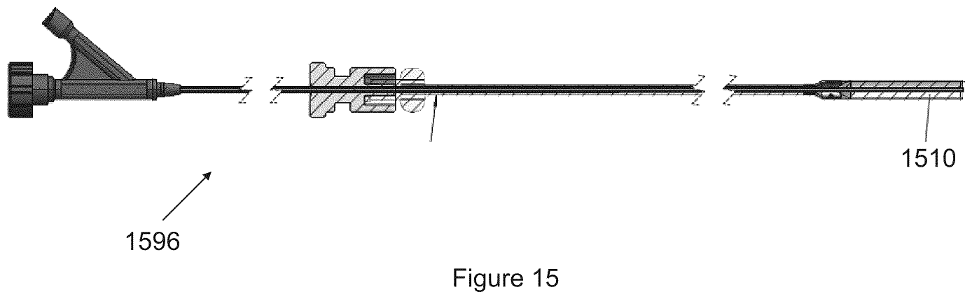

Referring now to FIG. 14, showing a schematic view of a pre-insertion implant delivery system 1400 including capsule 1410 optionally defined by an outer sheath with a cylindrical and/or substantially cylindrical shape, holding a crimped implant 1425, tabs 1435 & 1440 and an implant holder 1416. In some embodiments, capsule 1410 is attached to a guidance unit (for example, as shown in FIG. 15) and/or another guidance element which is connected and/or comprises a guide (e.g., guide wire tube 415) at a proximal end of capsule 1410 and has an open end at the distal end thereof, for implant passage therethrough. In some embodiments, the open end has a cross-section area of at least 70% of a maximal outer diameter of delivery system 1400. The guidance element is used for directing the insertion of implant 1452 to a desired deployment site (e.g., lumenal surface). For example, the guidance element can be used for directing a cylindrical self-expanding body sized to be implanted in a pulmonary artery, to the pulmonary artery.

In the figure, one notes that the end 1495 is open and does not make use of a distal conical tip (e.g., a nose cone) as is common in delivery systems. In some embodiments the guide, (such as guide 415) (not shown for clarity purposes) may protrude beyond the end 1495 but there is no need for a nose cone over the end 1495 of the outer sheath of capsule 1410 to close the capsule. The lack of a distal conical tip has the potential advantage of reducing and/or avoiding the risk of the nose cone dislodging the deployed implant while withdrawing the nose cone through the implant during retraction of the delivery system. The lack of a nose cone may have a particular use for an implant that forms a diametrical constriction, as shown for example in FIG. 3, in which the risk of dislodgement during retraction of the nose cose is greater.

In some embodiments, first, a guide sheath is introduced until it reaches the deployment site and then delivery system 1400 (e.g., capsule 1410) is inserted therethrough. Using a guide sheath has the potential advantage of reducing and/or avoiding trauma to surrounding tissue and/or vasculature, for example, caused by a sharp edge of the capsule advanced without a guide sheath. Upon reaching the deployment site, first, the guide sheath is retracted to reveal capsule 1410 (and/or capsule 1410 is pushed through the guide sheath), and then the deployment is performed by retracting the capsule to expose the implant.

As noted, the delivery unit is generally associated with and/or comprises a guide (e.g., guide wire tube), optionally, the guide is part of an implant insertion guidance unit. FIG. 15 shows such a guidance unit 1596 with implant capsule 1510 including crimped implant (not visible) in the capsule 1510 and present and ready for delivery to an artery and/or other lumenal surface.

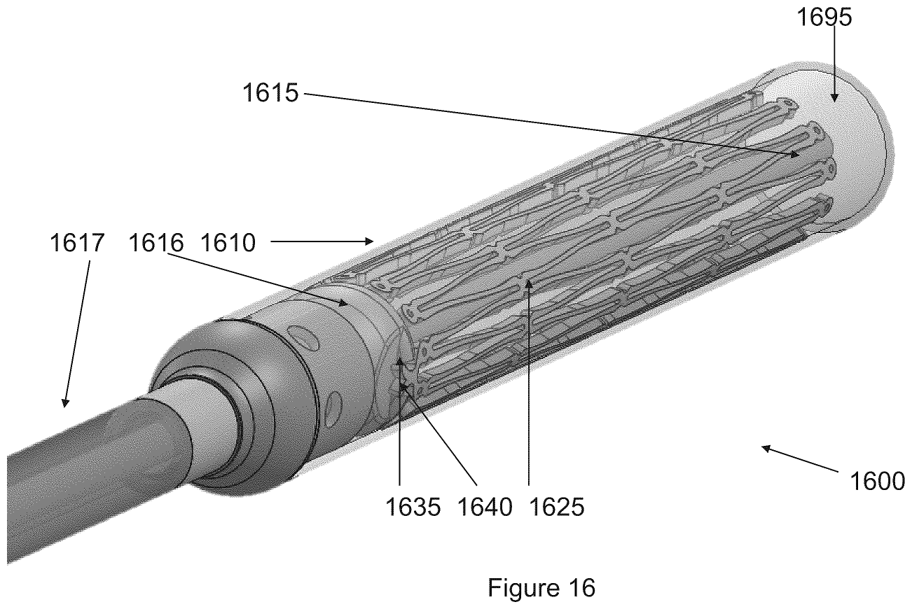

Referring now to FIG. 16, an implant delivery system 1600 includes a catheter capsule 1610 with a crimped implant 1625 held in place with tabs 1635 & 1640 attached to an implant holder 1616, such that the implant is crimped over guide wire tube 1615 that is optionally under control of a doctor during implant 1625 delivery. There is no nose cone or tip at the distal end 1695 of the instant embodiment. In some embodiments, a catheter shaft 1617 is attached to catheter capsule 1610, such that retraction of catheter shaft 1617 retracts catheter capsule 1610 and allows the implant to expand. Until the retraction of catheter shaft 1617, the implant is held from moving back by implant holder 1616 which is mounted over guidewire tube 1615 and which is held stationary (e.g., both guidewire tube 1615 and implant holder 1616) during said retraction of the catheter shaft 1617. In some embodiments, guidewire tube 1615 passes axially and optionally concentrically within catheter shaft 1617, as shown for example in FIG. 16.

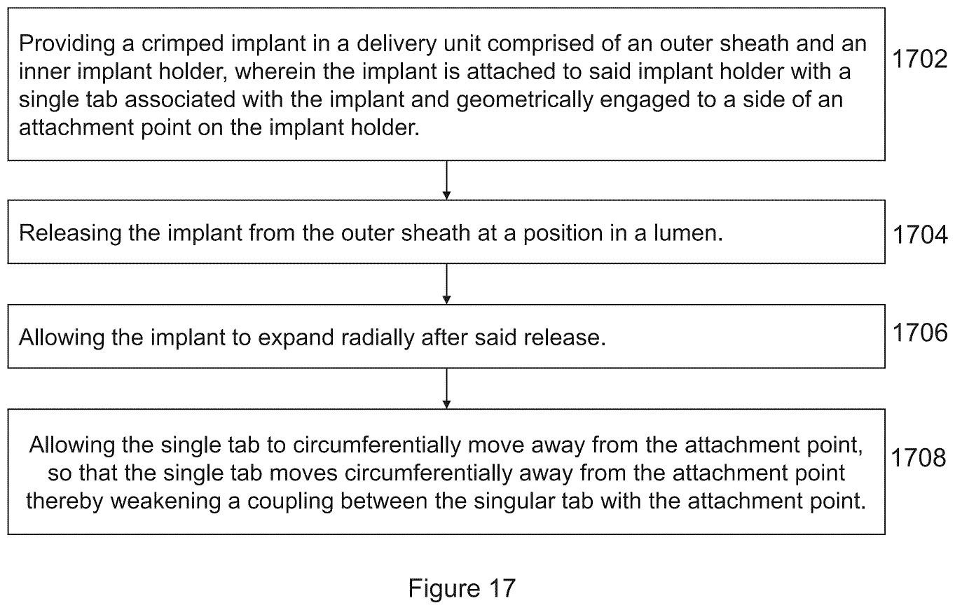

FIG. 17 shows a flowchart for an additional method of the invention. The invention includes a method for releasing an implant from a delivery unit into a lumen, including: providing a crimped implant in a delivery unit comprised of an outer sheath and an inner implant holder, wherein the implant is attached to said implant holder with a single tab associated with the implant and geometrically engaged to a side of an attachment point on the implant holder (702); releasing the implant from the outer sheath at a position in a lumen (704); allowing the implant to expand radially after said release (706); and, allowing the single tab to circumferentially move away from the attachment point, so that the single tab moves (circumferentially, radially, and/or axially relative to the attachment point) away from the attachment point thereby weakening a coupling between the singular tab with the attachment point (708).

In one aspect of the method, there is further including rotating the guide wire tube (e.g., guide 415, 1615) associated with the implant holder. In another aspect of the method, the single tab is realized as a plurality of single tabs.

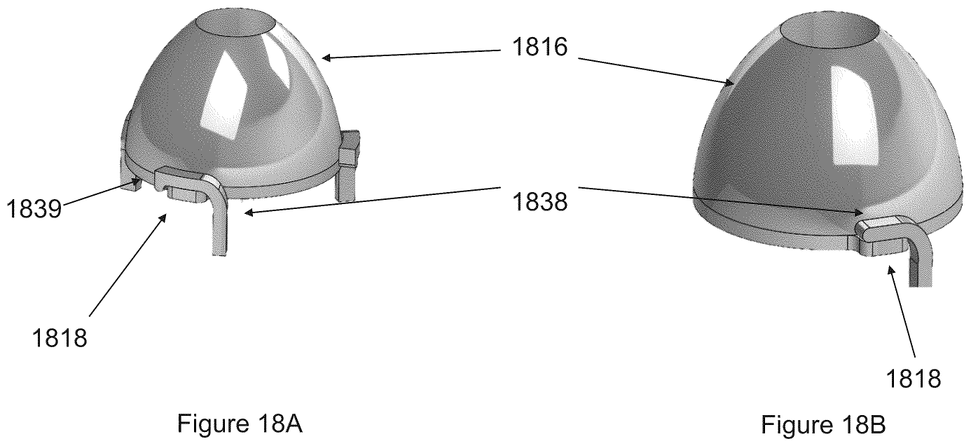

Attention is turned to FIG. 18A which shows a schematic view of an embodiment of the invention. A single tab 1838 is associated with an attachment point 1818 on an implant holder 1816. Optionally, attachment point 1818 is defined by a single geometry, such as a single protrusion. The single tab 1838 differs from prior art tabs in that its association with attachment point 1818 is on one side. In this embodiment, a plurality of single tabs 1838 are associated with a plurality of implant crowns and attachment points 1818. Typically, 2, 4, or 6 single tabs 1838 are employed. To help release of the single tab 1838, the guidewire tube (for example guide 415, shown in FIG. 4A) might be rotated to facilitate disassociation of single tab 1838 and implant holder 1816. Rotating the guidewire tube moves (e.g., rotates) implant holder 1816 and potentially prompts disassociation of single tab 1838 and attachment point 1818. FIG. 18B shows an alternative version of a single tab 1838 and its association with an attachment point 1818 connected to an implant holder 1816. Self-expansion of the Nitinol (or similar material) causes release of most of the tabs 1838. Even if a single tab 1838 is still attached to attachment point 1818, its release will be most likely. FIG. 18A shows a small portion of the tab 1838 wrapped around the attachment point 1818. This overhang 1839 is generally between 0.1 and 2 times the axial thickness of the attachment point, and preferably less than 1.

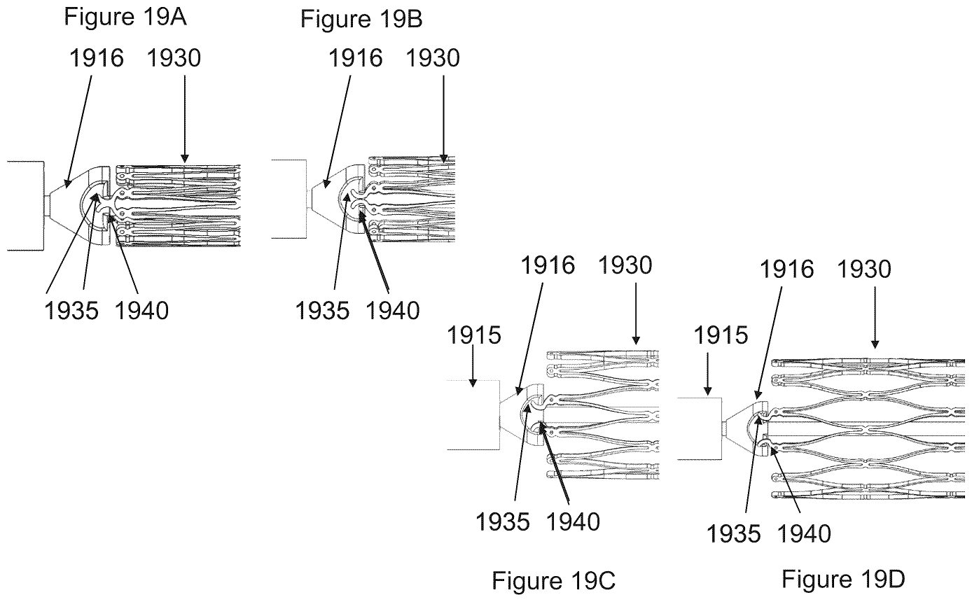

Attention is turned to FIGS. 19A, 19B, 19C and 19D, which show in self-expansion of a Nitonil-based implant 1930 and the concomitant release of first tab 1935 and second tab 1940 from associated implant holder 1916. In FIGS. 19C & 19D, the first tab 1935 and second tab 1940 are no longer in physical contact with the attachment points (not visible in these figures) on the deployment arm 1915.

It is expected that during the life of a patent maturing from this application many relevant lumen implant delivery units will be described; the scope of the term implant delivery unit is intended to include all such new technologies a priori. It is understood that additional devices and methods combining elements of the above embodiments may be employed without straying from the spirit of the invention.

As used herein with reference to quantity or value, the term “about” means “within 20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A delivery unit, comprising:
a capsule including an outer sheath and an inner implant holder having at least one attachment point for attaching an implant thereto;
an implant, comprising:
a first tab associated with a first circumferential location of said implant, wherein said first tab is sized and shaped for geometric engagement with said attachment point; and,
a second tab associated with a second circumferential location of said implant and sized and shaped for geometric engagement with said attachment point,
wherein said implant has a first, collapsed state within said sheath and a second, expanded state outside said sheath and wherein said first location and said second location are further circumferentially apart in said expanded state than in said collapsed state,
wherein said first tab and said second tab hold said implant to said attachment point in said first, collapsed, state, and move circumferentially apart in said second, expanded, state, thereby weakening an engagement of said tabs to said attachment point.

2-35. (canceled)

36. The delivery unit according to claim 1, wherein said implant includes at least one stent, and wherein said at least one stent is made, in part, of a self-expanding material.

37. The delivery unit according to claim 1, comprising one or more additional first tab and additional second tab, provided as pairs, wherein each tab is located on a unique position on said implant.

38. The delivery unit according to claim 1, wherein said implant holder comprises a retractable deployment arm and said at least one attachment point comprises a plurality of attachment points.

39. The delivery unit according to claim 36, wherein said first tab and said second tab are associated with crown-shaped structures at an end of said stent.

40. The delivery unit according to claim 1, wherein said first tab and said second tab are identical in shape or mirrored.

41. The delivery unit according to claim 1, wherein said first tab and said second tab have a rounded feature.

42. The delivery unit according to claim 1, wherein said first tab and said second tab have a rectangular feature.

43. The delivery unit according to claim 1, wherein said first tab and said second tab face each other around said attachment point.

44. The delivery unit according to claim 1, wherein said first tab and said second tab are associated with different geometries of the attachment point.

45. The delivery unit according to claim 1, wherein said capsule has an open end at a distal end thereof, for implant passage therethrough.

46. The delivery unit according to claim 45, wherein the distal end lacks a conical tip.

47. The delivery unit according to claim 1, comprising a guide that protrudes beyond the open distal end of said capsule, wherein said guide passes through said inner implant holder, and wherein said implant is crimped over said guide and within said capsule.

48. The delivery unit according to claim 1, comprises a balloon, associated with said implant holder, and positioned adjacent to said first tab and said second tab, such that inflation of said balloon forces the separation of said first tab and said second tab.

49. A method for releasing an implant from a delivery unit into a lumen, including:
providing a crimped implant in a delivery unit comprised of an outer sheath and an inner implant holder, wherein said implant is attached to said implant holder with a first tab and a second tab associated with said implant and geometrically engaged to a same attachment point on said implant holder;
releasing said implant from said outer sheath at a position in a lumen;
allowing said implant to expand radially after said release; and,
allowing said first tab and said second tab to circumferentially move away from each other, so that at least one tab moves circumferentially away from said attachment point thereby weakening a coupling between said first tab and said second tab with said attachment point.

50. The method according to claim 49, wherein said lumen is an artery.

51. The method according to claim 49, further including fluoroscopic monitoring of positions of said first tab and said second tab after said allowing said implant to expand.

52. The method according to claim 49, wherein said implant is made, in part, of a self-expanding material, and wherein the method further including expanding a balloon associated with said implant holder after said releasing.

53. The method according to claim 49, wherein one or both holds true:
said releasing is initiated by moving said inner implant holder out of said outer sheath; and
said releasing is initiated by a rotation of said implant holder.

54. A method for releasing an implant from a delivery unit into a lumen, including:
providing a crimped implant in a delivery unit comprised of a capsule and an inner implant holder, wherein said implant is attached to said implant holder with a single tab associated with said implant and geometrically engaged to a side of an attachment point on said implant holder;
releasing said implant from said capsule at a position in a lumen;
allowing said implant to expand radially after said release; and,
allowing said single tab to circumferentially move away from said attachment point, so that said single tab moves circumferentially away from said attachment point thereby weakening a coupling between said single tab with said attachment point.
